(19) United States
(12) Patent Application Publication
(10) Pub. No.: US 2010/0152480 A1
(43) Pub. Date: Jun. 17, 2010

(54) PROCESS FOR SYNTHESIS OF CATIONIC SURFACTANTS

(76) Inventor: Ghare Vishwas Sadhu, Thane (W) (IN)

Correspondence Address: LUCAS & MERCANTI, LLP, 475 PARK AVENUE SOUTH, 15TH FLOOR, NEW YORK, NY, 10016, US

(21) Appl. No.: 12/636,083

(22) Filed: Dec. 11, 2009

(30) Foreign Application Priority Data
Dec. 16, 2008 (IN) 2616/MUM/2008

(51) Int. Cl. C07C 229/30

(52) U.S. Cl. 560/169

(57) ABSTRACT

The present invention relates to the methods of synthesizing hydrochloride salt of N-fatty acyl substituted amino acid ethyl ester which comprises a) condensation of aqueous solution of esterified amino acid with an acid halide to obtain an intermediate suspension and b) isolating a hydrochloride salt of N-fatty acylsubstituted amino acid ethyl ester from the intermediate suspension.

### FIELD OF THE INVENTION

The present invention relates to a process for synthesis of cationic surfactants. Particularly the invention relates to processes for synthesis of hydrochloride salt of N-fatty acylsubstituted amino acid ethyl esters.

### BACKGROUND OF THE INVENTION

Ethyl lauroyl arginate hydrochloride (ELA) is derived from naturally occurring substances, L-arginine and lauric acid. It has cationic surfactant properties. It also has antimicrobial activity against bacteria, algae and fungi. ELA acts by modifying the permeability of cell membranes of living organisms. It is envisaged that it would be used as a multi functional component in the formulation of cosmetic products.

The active ingredient of ethyl lauroyl arginate is the hydrochloride salt of an N-fatty acyl-substituted amino acid ethyl ester, ethyl-Na-lauroyl-L-arginate HCl. Ethyl lauroyl arginate is a white powder and its solubility in water at 20° C. is greater than 247 g/kg.

Chemical name: ethyl-Na-dodecanoyl-Larginate HCl

CAS number: 60372-77-2

Molecular weight: 421.02.

Ethyl lauroyl arginate contains this active ingredient in the range between 85-95%.

Structure: Ethyl N-lauroyl-L-arginate HCl

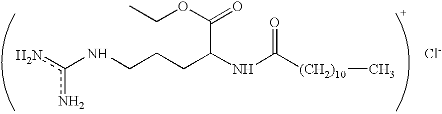

ELA is known to be effective against the gram positive bacteria (Arthrobacter oxydans, Bacillus cereus var mycoide, Bacillus subtilis, Bacillus megaterium, Clostridium perfringens, Clostridium perfringens, Listeria monocytogenes, Staphylococcus aureus, Micrococcus luteus, Lactobacillus delbrueckii ssp lactis), gram negative bacteria (Alcaligenes faecalis, Bordetella bronchiseptica, Citrobacter freundii, Citrobacter intermedium, Enterobacter aerogenes, Escherichia coli, Escherichia coli, Klebsiella pneumoniae var pneumoniae, Proteus mirabilis, Pseudomonas aeruginosa, Salmonella typhimurium, Serratia marcenses, Mycobacterium phlei) and also effective against moulds and yeast (Aspergillus niger, Aureobasidium pullulans, Cladosporium cladosporioides, Gliocadium virens, Chaetonium globosum, Penicillium chrysogenum, Penicillium fimiculosum, Penicillium camemberti, Candida albicans, Rhodotorula rubra, Saccharomyces cerevisiae).

Ethyl lauroyl arginate hydrochloride and its hydrolysis products have been sufficiently characterized to assure safe human consumption. Because of its antimicrobial properties, cationic surfactant activity and due to its non-toxicity it is useful as surfactant, antimicrobial and anti-static agent in cosmetic and toiletry formulations such as soaps, oral care products, deodorants, anti-dandruff shampoos. It is also useful as a preservative in the food industry.

Methods of synthesis of ethyl lauroyl arginate hydrochloride have been reported in several patents/published patent applications.

Following patents/applications disclose methods of synthesis of ethyl lauroyl arginate hydrochloride

Patent ES512643 discloses a process for synthesis of ethyl lauroyl arginate hydrochloride in two steps. The first step is the esterification of basic amino acid with alcohol, using thionyl chloride as a catalyst to give esterified amino acid and the second step is the condensation of an esterified amino acid derivative with a fatty acid as a free acid in the presence of coupling reagent such as dicyclohexylcarbodiimide (DCC). Furthermore, the first step of esterification involves heating for about 16 hours.

In another, Patent EP0749960 a two-step process for preparation of ethyl lauroyl arginate hydrochloride is disclosed. The first step is the esterification of amino acid with alcohol in presence of thionyl chloride as a catalyst. Step one is differing from step one disclosed in ES-A-512643 by providing in the first step a dispersion of the basic type amino acid in alcohol and adding catalyst like thionyl chloride to this dispersion in drop-wise manner. A further difference is that the second step i.e. the condensation of an esterified amino acid derivative is carried out with fatty acid halide instead of fatty acid. Furthermore, the second step of condensation of esterified amino acid is performed in aqueous alkaline medium at pH about 8-10.

Further, U.S. Pat. No. 7,087,769 discloses a process for the preparation of cationic surfactants derived from the condensation of fatty acid chlorides with esterified amino acids in an aqueous medium in the pH range of 6 and 6.9.

The yields obtained in these prior art processes is low. Further the aforesaid processes do not provide a comparatively pure product. Still further these processes are lengthy, time consuming and also quite expensive. Also some of these processes use toxic and corrosive reagents and are not safe. There is therefore a need to have an improved process over the prior art to produce a hydrochloride salt of an N-fatty acylsubstituted amino acid ethyl ester on the commercial scale, with improved yield and purity and which is economical and safe.

### OBJECTS OF THE INVENTION

It is an object of the present invention to provide processes for the synthesis of hydrochloride salt of N-fatty acylsubstituted amino acid ethyl esters with improved yield and purity.

Another object of the present invention is to provide processes for the synthesis of hydrochloride salt of N-fatty acylsubstituted amino acid ethyl esters, which employ nontoxic starting materials and catalysts.

Yet another object of the present invention is to provide processes for the synthesis of hydrochloride salt of N-fatty acylsubstituted amino acid ethyl esters, which are simple, efficient and cost effective.

Still another object of the present invention is to provide processes for the synthesis of hydrochloride salt of N-fatty acylsubstituted amino acid ethyl esters, which are safe.

### SUMMARY OF THE INVENTION

In accordance with the present invention there is provided a method for the synthesis of hydrochloride salt of N-fatty acylsubstituted amino acid ethyl ester; said method comprising the following steps:
A. condensation of aqueous solution of esterified amino acid with an acid halide to obtain an intermediate suspension comprising the following steps:
   a. dissolving esterified amino acid in distilled water with continuous stirring for about 10 minutes to obtain a clear solution;
   b. lowering the temperature of the solution to about 5° C. to 10° C. to obtain a cooled solution;
   c. adjusting the pH of the cooled solution in the range of about 7 to 7.9 by adding sodium hydroxide solution; and
   d. condensing the solution by simultaneously adding an acid halide and sodium hydroxide solution with continuous stirring at a temperature of about 5° C. to 10° C. for about 3 hours to obtain a intermediate suspension;
B. isolating a hydrochloride salt of N-fatty acylsubstituted amino acid ethyl ester from the intermediate suspension.

In accordance with the first embodiment of the present invention the method step of isolating a hydrochloride salt of N-fatty acylsubstituted amino acid ethyl ester from the intermediate suspension comprising the following steps:
a) filtering the resultant intermediate suspension under vacuum to obtain a precipitate of hydrochloride salt of an N-fatty acylsubstituted amino acid ethyl ester;
b) washing the precipitate of hydrochloride salt of an N-fatty acylsubstituted amino acid ethyl ester with distilled water; and
c) drying the precipitate in a vacuum oven at a temperature of about 35° C. to 40° C. for a period of about 3 to 4 hours to obtain a hydrochloride salt of N-fatty acylsubstituted amino acid ethyl ester.

In accordance with the second embodiment of the present invention the method step of isolating a hydrochloride salt of N-fatty acylsubstituted amino acid ethyl ester from the intermediate suspension comprising the following steps:
a) adding the organic solvent to the intermediate suspension with continuous stirring to obtain a mixture;
b) heating the mixture at a temperature of about 30° C. to 35° C. to obtain a resultant mixture containing organic phase and aqueous phase;
c) separating the organic phase and the aqueous phase of the mixture by settling the mixture at a temperature of about 25° C. to 35° C.; and
d) distilling the organic phase under vacuum at a temperature of about 40° C. to 50° C. to obtain a hydrochloride salt of an N-fatty acylsubstituted amino acid ethyl ester.

In accordance with the third embodiment of the present invention the method step of isolating a hydrochloride salt of N-fatty acylsubstituted amino acid ethyl ester from the intermediate suspension comprising the following steps:
a) maintaining the pH of the intermediate suspension in the range of about 7 to 7.9 by addition of sodium hydroxide solution for about 1 hr at a temperature below 15° C. to obtain a first slurry;
b) filtering the first slurry to obtain a residue followed by washing with the distilled water under high vacuum;
c) drying the residue in the air for about 12 hrs;
d) adding the dried residue to ethyl acetate to obtain a mixture followed by heating the mixture to dissolve the residue in ethyl acetate;
e) adding a charcoal to the heated mixture followed by stirring for about 1 hour;
f) filtering the charcoal at a temperature of about 25° C. to 35° C. to obtain a filtrate;
g) cooling the obtained filtrate at a temperature below 10° C. to form a second slurry containing precipitated solid;
h) filtering the second slurry under vacuum to obtain a wet cake;
i) adding the wet cake to hexane to obtain a third slurry followed by stirring the third slurry for about 2 hours;
j) filtering the third slurry under high vacuum to obtain a solid hydrochloride salt of an N-fatty acylsubstituted amino acid ethyl ester; and
k) drying the solid hydrochloride salt in air followed by drying in a vacuum oven at room temperature till a constant weight is obtained.

Typically, the amino acid is selected from a group consisting of L-lysine, L-arginine and histidine.

Preferably, the amino acid used is L-arginine.

Typically, the acid halide is at least one selected from a group consisting of fatty acid halides and hydroxy acid halides.

Typically, the acid halide is fatty acid chloride.

Typically, the fatty acid chloride is at least one selected from a group consisting of chlorides of lauric acid, caprylic acid, capric acid, myristic acid and palmitic acid.

Typically, the fatty acid chloride is lauroyl chloride.

Typically, the organic solvent is at least one selected from a group of solvents consisting of ethyl acetate, methyl acetate, butyl acetate and ethyl formate.

### BRIEF DESCRIPTION OF THE ACCOMPANYING DRAWINGS

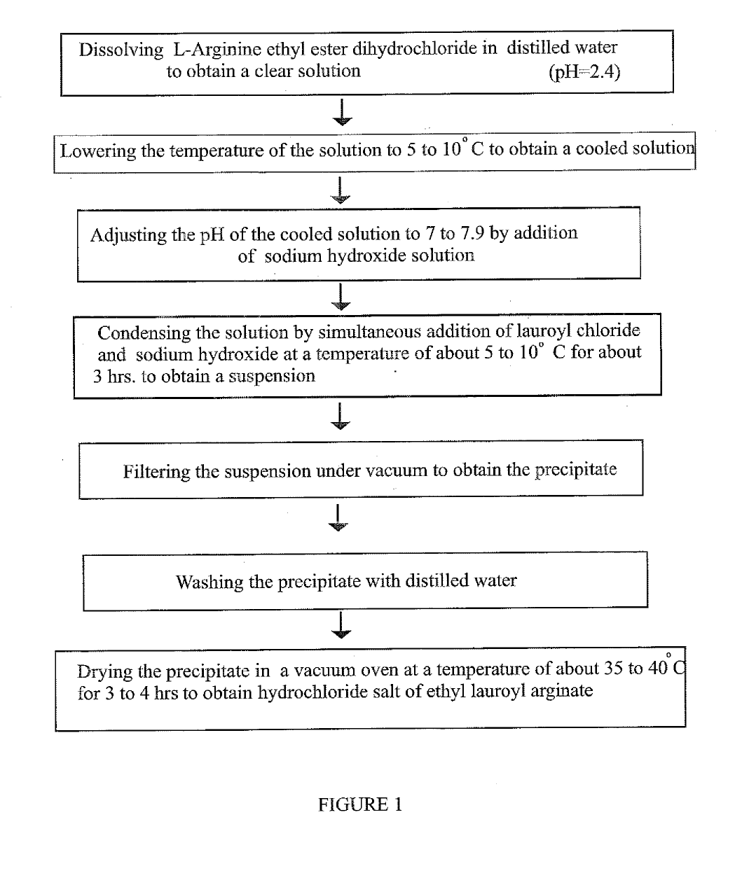

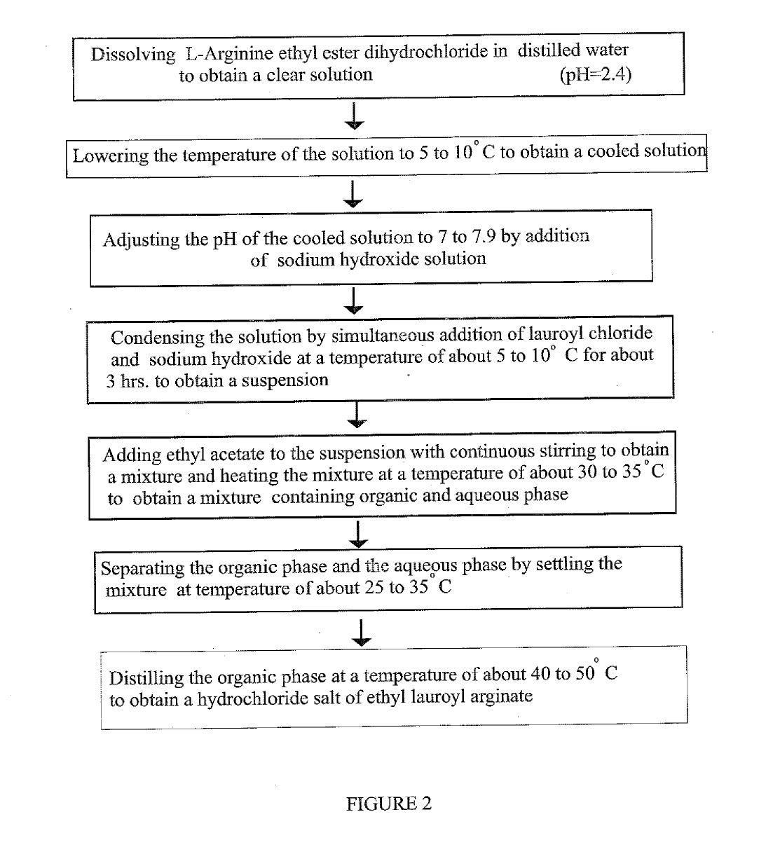

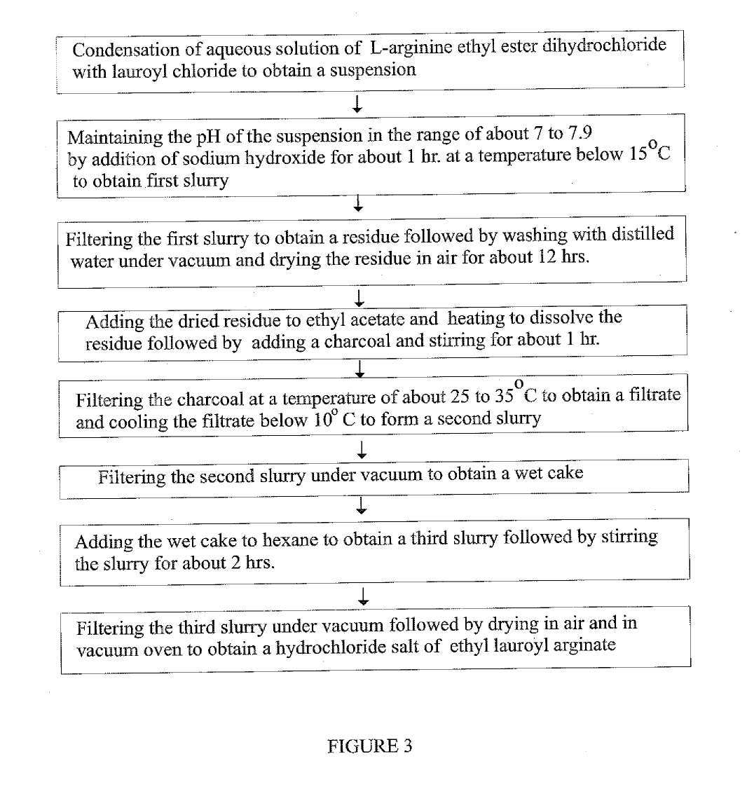

The invention will now be described with reference to accompanying drawings, in which, FIG. 1, FIG. 2 and FIG. 3 show a flowchart of the processes for preparation of ethyl lauroyl arginate hydrochloride in accordance with the present invention. The flow charts are self-explanatory.

Referring to the drawing, FIG. 1 shows a flowchart of the process for manufacturing of ethyl lauroyl arginate hydrochloride in accordance with the first embodiment of the present invention.

Referring to the drawing, FIG. 2 shows a flowchart of the process for manufacturing of ethyl lauroyl arginate hydrochloride in accordance with the second embodiment of the present invention.

Referring to the drawing, FIG. 3 shows a flowchart of the process for manufacturing of ethyl lauroyl arginate hydrochloride in accordance with the third embodiment of the present invention.

### DETAILED DESCRIPTION OF THE INVENTION

In accordance with the first embodiment of the present invention, there is provided a method for the synthesis of hydrochloride salt of N-fatty acylsubstituted amino acid ethyl ester; said method comprising the following steps:
A. condensation of aqueous solution of esterified amino acid with an acid halide to obtain an intermediate suspension comprising the following steps:
   a. dissolving esterified amino acid in distilled water with continuous stirring for about 10 minutes to obtain a clear solution;
   b. lowering the temperature of the solution to about 5° C. to 10° C. to obtain a cooled solution;
   c. adjusting the pH of the cooled solution in the range of about 7 to 7.9 by adding sodium hydroxide solution; and
   d. condensing the solution by simultaneously adding an acid halide and sodium hydroxide solution with continuous stirring at a temperature of about 5° C. to 10° C. for about 3 hours to obtain a intermediate suspension;
B. isolating a hydrochloride salt of N-fatty acylsubstituted amino acid ethyl ester from the intermediate suspension comprising the following steps:
   a. filtering the resultant intermediate suspension under vacuum to obtain a precipitate of hydrochloride salt of an N-fatty acylsubstituted amino acid ethyl ester;
   b. washing the precipitate of hydrochloride salt of an N-fatty acylsubstituted amino acid ethyl ester with distilled water; and
   c. drying the precipitate in a vacuum oven at a temperature of about 35° C. to 40° C. for a period of about 3 to 4 hours to obtain a hydrochloride salt of N-fatty acylsubstituted amino acid ethyl ester.

In accordance with the second embodiment of the present invention, there is provided a method for the synthesis of hydrochloride salt of N-fatty acylsubstituted amino acid ethyl ester; said method comprising the following steps:
A. condensation of aqueous solution of esterified amino acid with an acid halide to obtain an intermediate suspension comprising the following steps:
   a. dissolving esterified amino acid in distilled water with continuous stirring for about 10 minutes to obtain a clear solution;
   b. lowering the temperature of the solution to about 5° C. to 10° C. to obtain a cooled solution;
   c. adjusting the pH of the cooled solution in the range of about 7 to 7.9 by adding sodium hydroxide solution; and
   d. condensing the solution by simultaneously adding an acid halide and sodium hydroxide solution with continuous stirring at a temperature of about 5° C. to 10° C. for about 3 hours to obtain a intermediate suspension;
B. isolating a hydrochloride salt of N-fatty acylsubstituted amino acid ethyl ester from the intermediate suspension comprising the following steps:
   a. adding the organic solvent to the intermediate suspension with continuous stirring to obtain a mixture;
   b. heating the mixture at a temperature of about 30° C. to 35° C. to obtain a resultant mixture containing organic phase and aqueous phase;
   c. separating the organic phase and the aqueous phase of the mixture by settling the mixture at a temperature of about 25° C. to 35° C.; and
   d. distilling the organic phase under vacuum at a temperature of about 40° C. to 50° C. to obtain a hydrochloride salt of an N-fatty acylsubstituted amino acid ethyl ester.

In accordance with the third embodiment of the present invention, there is provided a method for the synthesis of hydrochloride salt of N-fatty acylsubstituted amino acid ethyl ester; said method comprising the following steps:
A. condensation of aqueous solution of esterified amino acid with an acid halide to obtain an intermediate suspension which comprising the following steps:
   a. dissolving esterified amino acid in distilled water with continuous stirring for about 10 minutes to obtain a clear solution;
   b. lowering the temperature of the solution to about 5° C. to 10° C. to obtain a cooled solution;
   c. adjusting the pH of the cooled solution in the range of about 7 to 7.9 by adding sodium hydroxide solution; and
   d. condensing the solution by simultaneously adding an acid halide and sodium hydroxide solution with continuous stirring at a temperature of about 5° C. to 10° C. for about 3 hours to obtain a intermediate suspension;
B. isolating a hydrochloride salt of N-fatty acylsubstituted amino acid ethyl ester from the intermediate suspension comprising the following steps:
   a. maintaining the pH of the intermediate suspension in the range of about 7 to 7.9 by addition of sodium hydroxide solution for about 1 hr at a temperature below 15° C. to obtain a first slurry;
   b. filtering the first slurry to obtain a residue followed by washing with the distilled water under high vacuum;
   c. drying the residue in the air for about 12 hrs;
   d. adding the dried residue to ethyl acetate to obtain a mixture followed by heating the mixture to dissolve the residue in ethyl acetate;
   e. adding a charcoal to the heated mixture followed by stirring for about 1 hour;
   f. filtering the charcoal at a temperature of about 25° C. to 35° C. to obtain a filtrate;
   g. cooling the obtained filtrate at a temperature below 10° C. to form a second slurry containing precipitated solid;
   h. filtering the second slurry under vacuum to obtain a wet cake;
   i. adding the wet cake to hexane to obtain a third slurry followed by stirring the third slurry for about 2 hours;
   j. filtering the third slurry under high vacuum to obtain a solid hydrochloride salt of an N-fatty acylsubstituted amino acid ethyl ester; and
   k. drying the solid hydrochloride salt in air followed by drying in a vacuum oven at room temperature till a constant weight is obtained.

In accordance with the present invention the amino acid used in the synthesis of hydrochloride salt of N-fatty acylsubstituted amino acid ethyl ester is selected from a group consisting of L-lysine, L-arginine and histidine.

Preferably, the amino acid used is L-arginine.

The acid halide used in the synthesis of hydrochloride salt of N-fatty acylsubstituted amino acid ethyl ester in accordance with the present invention is at least one selected from a group consisting of fatty acid halides and hydroxy acid halides.

Typically, the acid halide used is fatty acid chloride.

Typically, the fatty acid chloride is at least one selected from a group consisting of chlorides of lauric acid, caprylic acid, capric acid, myristic acid and palmitic acid.

Preferably, the fatty acid chloride used is lauroyl chloride.

The organic solvent used in the synthesis of hydrochloride salt of N-fatty acylsubstituted amino acid ethyl ester in accordance with the present invention is at least one selected from a group of solvents consisting of ethyl acetate, methyl acetate, butyl acetate and ethyl formate.

Following examples illustrate the invention, but are not intended to limit the scope of the present invention.

### EXAMPLES

### Example 1

47.1 gm of L-arginine ethyl ester dihydrochloride was transferred to a flask containing 300 gm of water and was stirred for 10 minutes to obtain a clear solution. This solution was cooled to 8° C. Further, 75 gm of 20% sodium hydroxide solution was taken in an addition funnel and 37.5 gm of lauroyl chloride was charged in another addition funnel. The pH meter electrode was dipped into the ethyl ester solution in the flask. The pH of the solution was 2.4. Then pH of the solution was adjusted to 7.5 by adding 20% sodium hydroxide solution. To this solution, lauroyl chloride and 20% sodium hydroxide were added simultaneously at a temperature of about 8° C. and the pH of the solution was maintained to 7.3. The addition of lauroyl chloride and 20% sodium hydroxide solution was carried out for 3 hours to obtain a solution containing thick white precipitate. The solution was then slowly allowed to attain the temperature of about 19° C. and held for 1 hour. The pH of the solution was 7.5. The pH of the solution was then adjusted to 7.3 by addition of few drops of 10% hydrochloric acid solution. The solution containing thick white precipitate was filtered under vacuum for 2 hours to obtain a white precipitate of ethyl lauroyl arginate hydrochloride which was then washed with 150 nil of distilled water. The white precipitate of ethyl lauroyl arginate hydrochloride thus obtained was dried in vacuum oven at the temperature of 35° C. to 40° C. for 4-5 hours.

(% Yield—94.92, HPLC purity—94.92%)

### Example 2

47.1 gm of L-arginine ethyl ester dihydrochloride was transferred to a flask containing 300 gm of water and was stirred for 10 minutes to obtain a clear solution. This solution was cooled to 8° C. Further, 75 gm of 20% sodium hydroxide solution was taken in an addition funnel and 37.5 gm of lauroyl chloride was charged in another addition funnel. The pH meter electrode was dipped into the ethyl ester solution in the flask. The pH of the solution was 2.4. The pH of the solution was adjusted to 7.5 by adding 20% sodium hydroxide solution. To the solution, lauroyl chloride and 20% sodium hydroxide were added simultaneously at the temperature of about 8° C. to 10° C. and the pH of the solution was maintained to 7.5. The addition of lauroyl chloride and 20% sodium hydroxide solution was carried out for 3 hours to obtain the solution containing trace white precipitate of (LAS). The solution containing thick white precipitate was then slowly allowed to attain the temperature of about 18° C. to 20° C. The pH of the solution was 7.3. A 250 gm of ethyl acetate was added to the above solution containing thick white precipitate with stirring and heated to a temperature of about 30° C. to get a mixture containing an organic phase and an aqueous phase. The mixture was held at the temperature of about 30° C. for 15 minutes. The mixture was allowed to settle and then separated. The organic phase was separated and the organic phase thus obtained was filtered at 25° C. to remove trace of the white precipitate. The filtered organic phase was charged in a distillation flask and distilled under vacuum at a temperature of about 50° C. to obtain a white crystalline hydrochloride salt of ethyl lauroyl arginate. (% Yield—98.10, HPLC purity—98.3%)

### Example 3

The process of example 1 was repeated except that pH of the solution was maintained to 5.7 and adjusted to 5.5 by addition of few drops of 10% hydrochloric acid solution. The obtained white precipitate of ethyl lauroyl arginate hydrochloride was dried for a period of 12 hours.

(% Yield—84, HPLC purity—91.65%)

### Example 4

The process of example 2 was repeated except that pH of the solution was maintained to 5.7. The product was isolated by addition of 250 gms of ethyl acetate solvent.

(% Yield—92.34, HPLC purity—95.3%)

### Example 5

The process of example 1 was repeated except that pH of the solution was maintained to 6.7 and adjusted to 6.5 by addition of few drops of 10% hydrochloric acid solution. The filtration was carried out for 5 hours and the white precipitate of ethyl lauroyl arginate hydrochloride obtained was dried in vacuum oven for a period of 9 hours.

(% Yield—88.1, HPLC purity—92.8%)

### Example 6

The process of example 2 was repeated except that pH of the solution was maintained to 6.7. The product was isolated by addition of 250 gms of ethyl acetate.

(% Yield—93.40, HPLC purity—95.6%)

### Example 7

The process of example 1 was repeated except that pH of the solution was maintained to 8.5 and adjusted to 6.5 by addition of few drops of 10% hydrochloric acid solution. The filtration was carried out for 2 hours and the white precipitate of ethyl lauroyl arginate hydrochloride obtained was dried in vacuum oven for a period of 4 hours.

(% Yield—86.9, HPLC purity—92.3%)

### Example 8

The process of example 2 was repeated except that pH of the solution was maintained to 8.5. The product was isolated by addition of 250 gms of ethyl acetate.

(% Yield—91, HPLC purity—94.92%)

### Example 9

The process of example 1 was repeated. The obtained white precipitate was further treated with 350 parts of n-hexane and stirred at 25° C. for 1 hour. The obtained product was isolated by filtration and washed with 50 parts of fresh n-hexane. The solid product was dried at 40° C. under vacuum for 3 hours.

(% Yield—91.9, HPLC purity—97.8%)

### Example 10

The process of example 2 was repeated. The obtained white precipitate was further treated with 350 parts of n-hexane and stirred at 25° C. for 1 hour. The obtained product was isolated by filtration and washed with 50 parts of fresh n-hexane. The solid product was dried at 40° C. under vacuum for 3 hours.

(% Yield—97.2, HPLC purity—99.1%)

### TECHNICAL ADVANCEMENT

The processes for the synthesis of ethyl lauroyl arginate as disclosed in the present invention provide high yield of ethyl lauroyl arginate as compared with the prior art processes. Further, the processes in accordance with the present invention provide a comparatively pure product. Still further the processes in accordance with the present invention are less time consuming compared to the prior art processes.

While considerable emphasis has been placed herein on the specific ingredients of the preferred processes, it will be appreciated that many additional ingredients can be added and that many changes can be made in the preferred processes without departing from the principles of the invention. These and other changes in the preferred processes of the invention will be apparent to those skilled in the art from the disclosure herein, whereby it is to be distinctly understood that the foregoing descriptive matter is to be interpreted merely as illustrative of the invention and not as a limitation.

1. A method of synthesizing hydrochloride salt of N-fatty acyl substituted amino acid ethyl ester; said method comprising the following steps:
A. condensation of aqueous solution of esterified amino acid with an acid halide to obtain an intermediate suspension comprising the following steps:
a. dissolving esterified amino acid in distilled water with continuous stirring for about 10 minutes to obtain a clear solution;
b. lowering the temperature of the solution to about 5° C. to 10° C. to obtain a cooled solution;
c. adjusting the pH of the cooled solution in the range of about 7 to 7.9 by adding sodium hydroxide solution;
d. condensing the solution by simultaneously adding an acid halide and sodium hydroxide solution with continuous stirring at a temperature of about 5° C. to 10° C. for about 3 hours to obtain a intermediate suspension;
B. isolating a hydrochloride salt of N-fatty acylsubstituted amino acid ethyl ester from the intermediate suspension.

2. The method as claimed in claim 1, wherein the method step of isolating a hydrochloride salt of N-fatty acylsubstituted amino acid ethyl ester from the intermediate suspension comprising the following steps:
a. filtering the resultant intermediate suspension under vacuum to obtain a precipitate of hydrochloride salt of an N-fatty acylsubstituted amino acid ethyl ester;
b. washing the precipitate of hydrochloride salt of an N-fatty acylsubstituted amino acid ethyl ester with distilled water; and
c. drying the precipitate in a vacuum oven at a temperature of about 35° C. to 40° C. for a period of about 3 to 4 hours to obtain a hydrochloride salt of N-fatty acylsubstituted amino acid ethyl ester.

3. The method as claimed in claim 1, wherein the method step of isolating a hydrochloride salt of N-fatty acylsubstituted amino acid ethyl ester from the intermediate suspension comprising the following steps:
a. adding the organic solvent to the intermediate suspension with continuous stirring to obtain a mixture;
b. heating the mixture at a temperature of about 30° C. to 35° C. to obtain a resultant mixture containing organic phase and aqueous phase;
c. separating the organic phase and the aqueous phase of the mixture by settling the mixture at a temperature of about 25° C. to 35° C.; and
d. distilling the organic phase under vacuum at a temperature of about 40° C. to 50° C. to obtain a hydrochloride salt of an N-fatty acylsubstituted amino acid ethyl ester.

4. The method as claimed in claim 1, wherein the method step of isolating a hydrochloride salt of N-fatty acylsubstituted amino acid ethyl ester from the intermediate suspension comprising the following steps:
a. maintaining the pH of the intermediate suspension in the range of about 7 to 7.9 by addition of sodium hydroxide solution for about 1 hr at a temperature below 15° C. to obtain a first slurry;
b. filtering the first slurry to obtain a residue followed by washing with the distilled water under high vacuum;
c. drying the residue in the air for about 12 hrs;
d. adding the dried residue to ethyl acetate to obtain a mixture followed by heating the mixture to dissolve the residue in ethyl acetate;
e. adding a charcoal to the heated mixture followed by stirring for about 1 hour;
f. filtering the charcoal at a temperature of about 25° C. to 35° C. to obtain a filtrate;
g. cooling the obtained filtrate at a temperature below 10° C. to form a second slurry containing precipitated solid;
h. filtering the second slurry under vacuum to obtain a wet cake;
i. adding the wet cake to hexane to obtain a third slurry followed by stirring the third slurry for about 2 hours;
j. filtering the third slurry under high vacuum to obtain a solid hydrochloride salt of an N-fatty acylsubstituted amino acid ethyl ester; and
k. drying the solid hydrochloride salt in air followed by drying in a vacuum oven at room temperature till a constant weight is obtained.

5. The method as claimed in of claim 1, wherein the amino acid is selected from a group consisting of L-lysine, L-arginine and histidine, Preferably, L-arginine.

6. The method as claimed in of claim 1, wherein the acid halide is at least one selected from a group consisting of fatty acid halides and hydroxy acid halides.

7. The method as claimed in of claim 6, wherein the acid halide is fatty acid chloride.

8. The method as claimed in of claim 7, wherein the fatty acid chlorides is at least one selected from a group consisting of chlorides of lauric acid, caprylic acid, capric acid, myristic acid and palmitic acid.

9. The method as claimed in of claim 8, wherein the fatty acid chloride is lauroyl chloride.

10. The method as claimed in of claim 3, wherein the organic solvent is at least one selected from a group of solvents consisting of ethyl acetate, methyl acetate, butyl acetate and ethyl formate.
